(19) United States
(12) Patent Application Publication
(10) Pub. No.: US 2009/0223892 A1
(43) Pub. Date: Sep. 10, 2009

(54) USE OF NITRATE SALTS FOR SUPPRESSING DISTURBING ODOURS

(76) Inventor: Stefan Vollmuth, Krefeld (DE)

Correspondence Address: FULBRIGHT & JAWORSKI, LLP, 666 FIFTH AVE, NEW YORK, NY, 10103-3198, US

(21) Appl. No.: 12/162,394

(22) PCT Filed: Feb. 1, 2007
(86) PCT No.: PCT/EP07/50970

§ 371 Date: Jul. 28, 2008

(30) Foreign Application Priority Data
Feb. 8, 2006 (DE) 10 2006 006 016.4

(51) Int. Cl. C02F 1/72; A61L 9/01

(52) U.S. Cl. 210/631; 210/757

(57) ABSTRACT

Nitrate salts are used to suppress disturbing biologically produced odors.

The present invention relates to the use of nitrate salts for suppressing disturbing biologically produced odors in the sanitary field.

The terms “suppressing” and “suppress” are to be understood as meaning “avoiding and/or eliminating” and “avoid and/or eliminate”, respectively.

The present invention relates in particular to the use of nitrate salts for suppressing the development of biological odors in the sanitary field, in particular in waste-pipes, drains, waste traps, pump units and/or in waste-water-carrying areas. The nitrate salts used are preferably metal nitrates, for example aluminum nitrate, aluminum nitrate sulfate, calcium nitrate, iron nitrate or a mixture of one or more of these substances.

In many households, but also in public buildings or industrial complexes, unpleasant odors form in waste-pipes, drains, waste traps, pump units and/or in waste-water-carrying areas. These odors generally come from anaerobic biological processes in the corresponding systems. In most cases the substances that produce the odor are organic sulfides or amines, but also hydrogen sulfide. The latter is a highly toxic gas with the very unpleasant odor of rotten eggs.

Such odor formation is traditionally eliminated by destroying the biofilm in the waste-water pipes by disinfection with chlorine bleach solution, sodium hydroxide solution and/or hydrogen peroxide. These chemicals have corrosive, irritating and oxidizing properties and can result in serious injury if not used properly.

Under anaerobic conditions, sulfate dissolved in the water is reduced by sulfate-reducing bacteria, with the formation of hydrogen sulfide. The sulfur of the sulfate thereby serves as electron acceptor for the bacteria. However, this process only takes place when the oxygen saturation falls below 20%. The anaerobic biological degradation of nitrate, on the other hand, begins as soon as the oxygen saturation falls below 80%, so that the nitrogen of the nitrate is available as electron acceptor for certain bacteria more readily and more rapidly than the sulfur of the sulfate. The presence of small amounts of nitrate therefore suppresses biogenic sulfate reduction and accordingly hydrogen sulfide formation. In addition, in the presence of nitrate, nitrate-reducing bacteria are especially preferred over sulfate-reducing bacteria in the biofilms. This leads to an increase in the nitrate-reducing bacteria and a decrease in the sulfate-reducing bacteria in the biofilm. This fact has been used very successfully for many years in sewer systems and closed systems for water treatment, for example in car washes, for suppressing the formation of biogenic hydrogen sulfide using calcium nitrate and nitrate-containing aluminum salts.

From EP-A-559272 there is further known a process in which nitrate is used for reducing hydrogen sulfide and other odor constituents in liquid manure. However, this process is based not on a biological reaction but on a chemical oxidation.

Also known are drain cleaners which, as well as containing alkali hydroxides and aluminum, also contain nitrate salts. An example of such a drain cleaner is Drano® Rohrfrei from Johnson Wax GmbH.

In this drain cleaner, however, nitrate is used for oxidation and accordingly for avoiding explosive hydrogen, which forms by the reaction of aluminum with water. The nitrate has no effect on the actual cleaning process and is not used to suppress disturbing biologically produced odors.

There has not hitherto been available for use on a very small scale, for example in the home, in commercial cleaning and in building management, a use of nitrate salts for suppressing disturbing biologically produced odors in waste-water-carrying areas that is comparable to the large-scale solution.

The object of the present invention is, therefore, to provide a process which suppresses disturbing biologically produced odors in the sanitary field, in particular in waste-pipes, drains, waste traps, pump units and/or in waste-water-carrying areas, and which can be used without difficulty on a very small scale, for example in the home, in commercial cleaning and in building management.

Surprisingly, this problem is solved according to the invention by the features of the main claim. Preferred embodiments are to be found in the dependent claims. In particular, the object is achieved by the use according to the invention of nitrate-containing mixtures for suppressing disturbing biologically produced odors.

In a use according to the invention, nitrate salts and/or aqueous solutions thereof are added as additive to commercial household cleaners.

In a further use according to the invention, acid-reacting nitrates, for example aluminum nitrate, aluminum nitrate sulfate and/or iron nitrate, and/or aqueous solutions thereof can replace acidic components conventionally used in cleaning agents, for example citric acid or acetic acid.

By regularly using the cleaning agents and washing them away with the waste water in drains and waste-water pipes, a sufficient amount of nitrate is always available to the biofilms so that the odor-forming processes are suppressed.

In a further use according to the invention, the nitrate salts or aqueous solutions thereof can also prevent odor formation if they are added to waste-pipes in small but regular amounts. Mixtures with biodispersing agents such as, for example, orange terpenes at the same time result in the avoidance of the growth of biofilms on the pipe walls. Mixtures with special bacterial cultures influence the biofilm so that nitrate-reducing bacteria are able to become established in the biofilm more quickly.

According to the invention, the polyaluminum nitrate sulfate marketed by Sachtleben Chemie GmbH under the trade mark NICASAL can be used as the nitrate-containing mixture for all the described uses according to the invention. This polyaluminum nitrate sulfate is disclosed in EP-A-1108679, or EP-B-1108679, the entirety of the disclosures of which form part of this description, and contains from 5.0 to 5.6 wt. % aluminum (Al³⁺), from 2.4 to 3.2 wt. % sulfate and from 14.5 to 25.0 wt. % nitrate. The polyaluminum nitrate sulfate can either be added as an additive to conventional cleaning agents or can be used as a replacement for acidic components, for example citric acid or acetic acid, or can be used directly for suppressing disturbing biologically produced odors.

Specifically, the present invention provides:
the use of nitrate salts for suppressing disturbing biologically produced odors in the sanitary field, in particular in waste-pipes, drains, waste traps, pump units and/or in waste-water-carrying areas, by suppressing biogenic sulfate reduction;
the use according to the invention of nitrate salts, wherein the nitrate salts are metal nitrates;
the use according to the invention of nitrate salts, wherein the metal nitrates are the nitrates of sodium, potassium, magnesium, calcium, aluminum, iron or mixtures of these nitrates;
the use according to the invention of nitrate salts, wherein the nitrate salts are added as additives to cleaning agents, preferably commercial cleaning agents;
the use according to the invention of nitrate salts, wherein the nitrate salts, preferably acid-reacting metal nitrates, particularly preferably aluminum nitrate, aluminum nitrate sulfate, iron nitrate or mixtures thereof, are used as a replacement for organic acids, preferably citric acid and/or acetic acid, and/or salts thereof in cleaning agents, preferably commercial cleaning agents;
the use according to the invention of nitrate salts, wherein the nitrate salts are used in the form of aqueous solutions;
the use according to the invention of nitrate salts, wherein the concentration of nitrate ions in the aqueous solutions is from 0.5 to 50 wt. %, preferably from 5 to 40 wt. %, particularly preferably from 10 to 35 wt. %;
the use according to the invention of nitrate salts, wherein from 70 to 90 wt. % of a cleaning agent are mixed with from 10 to 30 wt. % of an aqueous aluminum nitrate solution;
the use according to the invention of nitrate salts, wherein the aluminum nitrate solution contains from 2 to 6 wt. % Al³⁺ and from 20 to 40 wt. %, preferably from 25 to 35 wt. %, NO₃⁻;
the use according to the invention of nitrate salts, wherein from 70 to 90 wt. % of a cleaning agent are mixed with from 10 to 30 wt. % of an aqueous aluminum nitrate sulfate solution;
the use according to the invention of nitrate salts, wherein the aluminum nitrate sulfate solution contains from 4 to 7 wt. % Al³⁺, from 12 to 24 wt. % NO₃⁻ and from 2 to 3.5 wt. % SO₄²⁻;
the use according to the invention of nitrate salts, wherein the nitrate-salt-containing solutions contain from 0.01 to 5 wt. %, preferably from 0.1 to 5 wt. %, particularly preferably from 0.5 to 2 wt. %, of at least one biodispersing agent, preferably orange terpene;
the use according to the invention of nitrate salts, wherein the mixtures of nitrate-salt-containing solutions with cleaning agents contain from 0.01 to 5 wt. %, preferably from 0.1 to 5 wt. %, particularly preferably from 0.5 to 2 wt. %, of at least one biodispersing agent, preferably orange terpene;
the use according to the invention of nitrate salts, wherein the nitrate-salt-containing solutions contain from 0.01 to 5 wt. %, preferably from 0.05 to 2 wt. %, particularly preferably from 0.1 to 1 wt. %, of an aqueous suspension of bacteria that degrade harmful substances;
the use according to the invention of nitrate salts, wherein the mixtures of nitrate-salt-containing solutions with cleaning agents contain from 0.01 to 5 wt. %, preferably from 0.05 to 2 wt. %, particularly preferably from 0.1 to 1 wt. %, of an aqueous suspension of bacteria that degrade harmful substances;
the use according to the invention of nitrate salts, wherein these are metered as such, in the form of aqueous solutions and/or in admixture with cleaning agents directly into drains, waste traps, pump units and waste water;
the use according to the invention of nitrate salts, wherein the metering concentration of the aqueous solutions of the nitrate salts or of the mixture of these solutions with cleaning agents in domestic applications is from 5 ml every 4 weeks to 25 ml per day, preferably from 5 ml per week to 10 ml per day, particularly preferably from 10 ml per week to 10 ml per day;
the use according to the invention of nitrate salts, wherein the nitrate salts, the solutions thereof and/or the corresponding mixtures with cleaning agents are used preventively to avoid odors.

The invention is explained in detail by the following examples, without being limited thereto:

### EXAMPLE 1a

Commercial bath cleaner consisting of 5.5 wt. % citric acid, 0.3 wt. % fragrances, 0.5 wt. % fatty alcohol polyglycol ether, 0.6 wt. % alkyl polyglucoside, 0.5 wt. % ethoxy alcohol and 0.23 wt. % potassium tripolyphosphate, and 92.37 wt. % water.

### EXAMPLE 1b

80 wt. % of a commercial bath cleaner according to Example 1a are mixed with 20 wt. % of an aqueous aluminum nitrate sulfate solution comprising 5.35 wt. % Al³⁺, 16.0 wt. % NO₃⁻ and 2.8 wt. % SO₄²⁻. The product is stable and exhibits a comparable cleaning performance to Example 1a.

### EXAMPLE 1c

Mixture based on the commercial bath cleaner according to Example 1a in which the citric acid and the water are replaced by an aqueous aluminum nitrate sulfate solution comprising 5.35 wt. % Al³⁺, 16.0 wt. % NO₃⁻ and 2.8 wt. % SO₄²⁻.

The composition contains: 0.3 wt. % fragrances, 0.5 wt. % fatty alcohol polyglycol ether, 0.6 wt. % alkyl polyglucoside, 0.5 wt. % ethoxy alcohol, 0.23 wt. % potassium tripolyphosphate, 5.24 wt. % Al³⁺, 15.7 wt. % NO₃⁻, 2.7 wt. % SO₄²⁻ and 74.23 wt. % water. The product is stable and exhibits a comparable cleaning performance to Example 1a.

### EXAMPLE 2a

Commercial WC cleaner consisting of 5.5 wt. % citric acid, 1.0 wt. % sodium alkylsulfonate (60%), 0.3 wt. % fragrances, 0.002 wt. % coloring, 0.6 wt. % xanthan and 92.6 wt. % water.

### EXAMPLE 2b

A commercial WC cleaner according to Example 2a is mixed with 20 wt. % of an aqueous aluminum nitrate sulfate solution comprising 5.35 wt. % Al³⁺, 16.0 wt. % NO₃⁻ and 2.8 wt. % SO₄²⁻. The product is stable and exhibits a comparable cleaning performance to Example 2a.

### EXAMPLE 2c

Mixture based on the commercial bath cleaner according to Example 2a in which 46.3 wt. % water are replaced by an aqueous aluminum nitrate sulfate solution comprising 5.35 wt. % Al³⁺, 16.0 wt. % NO₃⁻ and 2.8 wt. % SO₄²⁻.

Composition: 1.0 wt. % sodium alkylsulfonate (60%), 0.3 wt. % fragrances, 0.002 wt. % coloring, 0.6 wt. % xanthan, 2.87 wt. % Al³⁺, 8.6 wt. % NO₃⁻, 1.5 wt. % SO₄²⁻ and 85.128 wt. % water. The product is stable and exhibits a comparable cleaning performance to Example 2a.

### EXAMPLE 3a

Product according to EP-A-1108679 comprising 5.35 wt. % Al³⁺, 16.0 wt. % NO₃⁻ and 2.8 wt. % SO₄²⁻.

### EXAMPLE 3b

Product according to EP-A-1108679 comprising 5.25 wt. % Al³⁺, 16.0 wt. % NO₃⁻, 2.8 wt. % SO₄²⁻ and 1 wt. % orange terpene.

### EXAMPLE 3c

Product according to EP-A-1108679 comprising 5.35 wt. % Al³⁺, 16.0 wt. % NO₃⁻, 2.8 wt. % SO₄²⁻ and 0.3 wt. % of an aqueous mixture of various bacterial cultures.

### EXAMPLE 3d

5 ml of the products according to Examples 3a, 3b and 3c are added every 4 weeks to the drain of a sink. The unpleasant odor was prevented over the period of the addition. When the addition is stopped, odor development occurs again after 6 to 8 weeks.

### EXAMPLE 3e

5 ml of the products according to Examples 3a, 3b and 3c are added weekly to a domestic pump unit to which a washing machine is connected. The unpleasant odor was prevented over the period of the addition. When the addition is stopped, odor development occurs again after 2 weeks.

1-18. (canceled)

19. A method comprising adding a sufficient amount of a nitrate salt to a waste-water-carrying area in an amount sufficient to suppress a biologically produced odor in the waste-water-carrying area.

20. The method of claim 19, wherein the nitrate salt is a metal nitrate.

21. The method according to claim 20, wherein the nitrate salt is a nitrate of sodium, potassium, magnesium, calcium, aluminum, iron or mixture thereof.

22. The method of claim 19, wherein the nitrate salt is added to a cleaning agent and forms a solution.

23. The method of claim 19, wherein the nitrate salt is aluminum nitrate, aluminum nitrate sulfate, iron nitrate or a mixture thereof and replaces an organic acid in a cleaning agent.

24. The method of claim 23, wherein the nitrate salt is provided in the form of and aqueous solution.

25. The method of claim 24, wherein the concentration of nitrate ion in the solution is from 0.5 to 50 wt. %.

26. The method of claim 19, wherein the nitrate salt is aluminum nitrate, and from 70 to 90 wt. % of a cleaning agent are mixed with from 10 to 30 wt. % of an aqueous solution of the aluminum nitrate.

27. The method of claim 26, wherein the aluminum nitrate solution contains from 2 to 6 wt. % Al³⁺ and from 20 to 40 wt. % NO₃⁻.

28. The method of claim 19, wherein the nitrate salt is ammonium nitrate sulfate, and from 70 to 90 wt. % of a cleaning agent are mixed with from 10 to 30 wt. % of an aqueous solution of the aluminum nitrate sulfate.

29. The method of claim 24, wherein the aluminum nitrate sulfate solution contains from 4 to 7 wt. % Al³⁺, from 12 to 24 wt. % NO₃⁻ and from 2 to 3.5 wt. % SO₄²⁻.

30. The method of claim 24, wherein the nitrate-salt-containing solution contain from 0.01 to 5 wt. % orange terpene.

31. The method of claim 22, wherein the mixtures of nitrate-salt-containing solution with cleaning agent contain from 0.01 to 5 wt. % orange terpene.

32. The method of claim 22, wherein the nitrate-salt-containing solution contains from 0.01 to 5 wt. % of an aqueous suspension of bacteria that degrade harmful substances.

33. The method of claim 22, wherein the mixtures of nitrate-salt-containing solution with cleaning agents contain from 0.01 to 5 wt of an aqueous suspension of bacteria that degrade harmful substances.

34. The method of claim 22, wherein the nitrate salts are metered in the form of aqueous solutions and/or in admixture with cleaning agents directly into drains, waste traps, pump units and waste water.

35. The method of claim, wherein the metering concentration in domestic applications is from 5 ml every 4 weeks to 25 ml per day of the solution is applied.

36. The method of claim 22, wherein the nitrate salt, the solution thereof and/or the corresponding mixtures with cleaning agents are preventively to avoid odors.
